(19) United States
(12) Patent Application Publication
(10) Pub. No.: US 2025/0232876 A1
(43) Pub. Date: Jul. 17, 2025

(54) COMPUTATIONAL MULTIPLEXING AND APPLICATION THEREOF

(71) Applicants: Emily D. Crawford, San Francisco, CA (US); Ryo Chijiiwa, Chico, CA (US)

(72) Inventors: Emily D. Crawford, San Francisco, CA (US); Ryo Chijiiwa, Chico, CA (US)

(73) Assignees: Emily D. Crawford, San Francisco, CA (US); Ryo Chijiiwa, Chico, CA (US)

(21) Appl. No.: 18/870,092

(22) PCT Filed: Jun. 2, 2023
(86) PCT No.: PCT/US2023/067898

§ 371 Date: Nov. 27, 2024

Related U.S. Application Data
(60) Provisional application No. 63/348,849, filed on Jun. 3, 2022.

(51) Int. Cl. G16H 50/20; C12Q 1/34; C12Q 1/6825; C12Q 1/6886; G06F 1/03; G16B 25/20

(52) U.S. Cl. CPC G16H 50/20; C12Q 1/34; C12Q 1/6825; C12Q 1/6886; G06F 1/03; G16B 25/20; C12Q 2600/156; G01N 2333/922

(57) ABSTRACT

Embodiments described herein relate to computational multiplexing and applications thereof. The disclosed method and system for performing multiplexed diagnostic testing include providing a sample comprising nucleic acid units to a plurality of channels of a multichannel device; introducing a unique set of probes and reporter moieties into each of the channels; detecting a first indicator readout from a set of channels, wherein the first indicator readout is generated by interaction of a probe within the unique set with one of the nucleic acid units in the sample; generating a readout code for the sample based at least in part on the first indicator readout detected from the first set of channels; matching the generated readout code against a plurality of readout code entries of a lookup table; and generating a detection result of the multiplexed diagnostic testing for the sample based on the matched readout code.

### CROSS REFERENCE TO RELATED APPLICATION

The present application claims the benefit of priority under 35 U.S.C. § 119(e) to U.S. Provisional Application No. 63/348,849, “COMPUTATIONAL MULTIPLEXING AND APPLICATION THEREOF”, filed on Jun. 3, 2022, which is currently co-pending herewith and which is incorporated by reference in its entirety.

### FIELD

The present disclosure generally relates to computational multiplexing, and in particular, relates to an application of computational multiplexing to physical multiplexing in multichannel assaying of biological identities.

### BACKGROUND

In clinical settings, a patient is often subjected to a large number of tests to receive a diagnosis. Typically, more than one sample (e.g., blood sample) from a patient are collected and subjected to separate tests for different (markers of) diseases/diagnostic targets. Some of the limitations to the current approach are the limited number of samples that can be reasonably collected from the patient and the cumulative cost of performing many separate tests, which often would prolong the timeline to receive the results. For some cases, collecting one sample from a patient may be sufficient if it can be physically divided into multiple smaller samples, where each can be fed into one of multiple channels, where each of the channels are then subjected to a separate test. In this multi-channel approach, the testing scheme may be limited by the amount of sample that can be collected, while the additive cost of the tests may still not be curtailed. Therefore, it is desirable to have a testing scheme with minimal individual tests but with which the sample can still be tested for a large number of different diagnostic targets or disease markers that can provide results in a shortened timeline with minimal incurred costs.

Such desirable testing schemes can be beneficial to all diagnostics areas, including for example, for infectious disease diagnostics, where it may be prudent to test for multiple pathogens at once, or for cancer diagnostics, where it may be desirable to test multiple oncogenic mutations in a single test. Thus, for the reasons provided above, there is a demand for a diagnosis scheme that includes a single test that enables simultaneous and/or high throughput detection and characterization of a large number of diagnostic targets or markers, pathogens, and/or genetic mutations, or many other biological identities.

### SUMMARY

In accordance with various embodiments disclosed herein, a method for performing multiplexed diagnostic testing is provided. The method includes providing a sample comprising nucleic acid units to a plurality of channels of a multichannel device; introducing a unique set of one or more probes and one or more reporter moieties into each of the plurality of channels; detecting a first indicator readout from a first set of channels of the plurality of channels, wherein the first indicator readout is generated by at least one of the one or more reporter moieties as a result of interaction of a probe within the unique set with one of the nucleic acid units in the sample; generating a readout code for the sample based at least in part on the first indicator readout detected from the first set of channels of the plurality of channels; matching the generated readout code against a plurality of readout code entries of a lookup table, wherein the plurality of readout code entries of the lookup table represent a plurality of biological identities associated with the nucleic acid units; and generating a detection result of the multiplexed diagnostic testing for the sample based on the matched readout code.

In accordance with various embodiments disclosed herein, a system for performing multiplexed diagnostic testing is provided. The system includes a multichannel device having a plurality of channels configured to receive a sample comprising nucleic acid units, wherein each of the plurality of channels are configured to receive a unique set of one or more probes and one or more reporter moieties into each of the plurality of channels, and wherein the multichannel device is configured to detect a first indicator readout from a first set of channels of the plurality of channels, wherein the first indicator readout is generated by at least one of the one or more reporter moieties as a result of interaction of a probe within the unique set with one of the nucleic acid units in the sample; and a processor communicatively coupled to the multichannel device and configured to perform multiplexing operations comprising: generating a readout code for the sample based at least in part on the first indicator readout detected from the first set of channels of the plurality of channels; matching the generated readout code against a plurality of readout code entries of a lookup table, wherein the plurality of readout code entries of the lookup table represent a plurality of biological identities associated with the nucleic acid units; and generating a detection result of the multiplexed diagnostic testing for the sample based on the matched readout code.

In accordance with various embodiments disclosed herein, a non-transitory machine-readable medium having stored thereon machine-readable instructions executable to cause a computing device to perform operations for generating a multiplexed diagnostic testing result is provided. The operations for generating the multiplexed diagnostic testing result include receiving a first indicator readout from a first set of channels of a plurality of channels of a multichannel device, wherein the first indicator readout is generated by interaction of one of nucleic acid units in a sample with a probe within a unique set of one or more probes in the plurality of channels of the multichannel device; generating a readout code for the sample based at least in part on the first indicator readout detected from the first set of channels of the plurality of channels; matching the generated readout code against a plurality of readout code entries of a lookup table, wherein the plurality of readout code entries of the lookup table represent a plurality of biological identities associated with the nucleic acid units; and generating the multiplexed diagnostic testing result for the sample based on the matched readout code.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 illustrates an example of a system 100 for performing multiplexed diagnostic testing, in accordance with various embodiments.

FIG. 2 illustrates an example testing scheme 200 for performing brute-force multiplexed diagnostic testing, in accordance with various embodiments.

FIG. 3 illustrates an example testing scheme 300 for performing multiplexed diagnostic testing, in accordance with various embodiments.

FIG. 4 illustrates an example testing scheme 400 for performing multiplexed diagnostic testing, in accordance with various embodiments.

FIG. 5 illustrates an example scenario 500 resulting from the testing scheme 400 for multiplexed diagnostic testing, in accordance with various embodiments.

FIGS. 6A, 6B, and 6C illustrate an example scenario 600a, 600b, and 600c, respectively, of data interpretations, in accordance with various embodiments.

FIGS. 7A and 7B illustrate an example scenario 700a and 700b, respectively, of data interpretations, in accordance with various embodiments.

FIGS. 8A and 8B illustrate tables 800a and 800b, respectively, from the results of an example coding scheme used in performing for multiplexed diagnostic testing, in accordance with various embodiments.

FIGS. 9A, 9B, and 9C illustrate how prevalence rates affect computational multiplexing results, in accordance with various embodiments.

FIG. 10 illustrates a table displaying results when prevalence rates are taken into account to improving test results, in accordance with various embodiments.

FIG. 11 illustrates a table 1100 displaying positive predictive values when various prevalence rate curves and error models are taken into account, in accordance with various embodiments.

FIG. 12 illustrates an example method S100 for performing multiplexed diagnostic testing, in accordance with various embodiments.

FIG. 13 is a block diagram illustrating a computer system 1300 with which embodiments of the disclosed systems and methods, or portions thereof may be implemented, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

One way to perform a single test that enables simultaneous and/or high throughput detection and/or characterization of a large number of diagnostic targets or markers is by multiplexing of the detection and/or characterization step. Such multiplexing can be achieved via physical multiplexing, for example, by collecting one sample from a patient and subjecting it to a test where different readouts correspond to different diagnostic targets. There are some limitations with this approach as the number of tests that can co-occur may be limited, which in turn, limits the number of readouts available in a given space. For example, in traditional polymerase chain reaction (PCR), multiplexing can be performed by targeting multiple targets in a single reaction. However, it is often difficult to multiplex in PCR for more than two or three targets in a single well where mixing assays with different chlorophores may be challenging. When more pairs of PCR primers are mixed together in a well, for example, all primers have to be in high concentrations. At higher concentrations of the primers in the well, there are more interactions between the different primers, which may cause unwanted primer-dimer couplings, which may out compete the intended interactions that one desires with multiplexing. Strategies to overcome this limitation may involve careful primer selection and/or enzymatic treatment to break up primer-dimer couplings to achieve a satisfiable PCR multiplexing.

As for quantitative PCR (qPCR), for example, as in a qPCR infectious disease assay where different fluorescent signals correspond to different pathogens that can be detected, typically no more than 5 markers or diagnostic targets can be multiplexed. Since at each fluorescent wavelength, the measurement may indicate that a given pathogen is either present or absent, the florescent space used in the qPCR can limit the number of markers. Anything more than approximately 5 different markers or targets may lead to the fluorescent signals overlapping, e.g., mixing, with one another. In other words, no more than approximately 5markers or diagnostic targets can be multiplexed in the qPCR space due to the limited number of non-overlapping readouts that can be extracted from the fluorescence space.

Another way of multiplexing can be achieved via genetic sequencing. In this approach, one sample can be collected from a patient and subjected to a test that results in genetic sequencing data which may correspond to various diagnostic targets. In a sequencing test for infectious diseases, for example, a large portion of the genetic sequencing data is typically compared to a list of known pathogen sequences, where a presence or an absence of a close match to a given pathogen genome may indicate whether that pathogen is present or absent. However, this approach is limited by the cost and the time to perform the genetic sequencing.

In summary, each of the methods described above is limited, to varying degrees, by sample availability and cost. In particular, molecular diagnostics (where DNA and/or RNA sequences are the analyte) lacks multiplexing methods that can identify greater than 5 markers or diagnostic targets simultaneously without incurring the high cost and time requirements of sequencing. With careful primer and probe design, it is possible to identify five different diagnostic targets per channel with qPCR. However, even if there are 20 channels, at most 100 markers or diagnostic targets can be evaluated in a given single qPCR test. Another approach that can be used for multiplexing signal detection and characterization is using a system known as clustered regularly interspaced short palindromic repeats (CRISPR). In CRISPR, an advantage compared to PCR or qPCR is that different CRISPR probes (called guide RNAs or gRNAs) do not physically interfere with each other during multiplexing. Thus, a CRISPR-based assay can be used to detect the presence of one or more of a large number of DNA (and/or RNA) sequences in a single sample, although it cannot distinguish which sequence(s) are present. Thus, the best currently available state of the art technologies still cannot perform a single test that is robust enough to reliably and reproducibly detect the presence of large numbers of unique DNA (and/or RNA) sequences in a single sample, which may be required for human diagnostics, pathogen surveillance, agriculture, veterinary diagnostics, or food safety programs, just to name a few. Thus, there is a need for a more advanced multiplex testing scheme that can be robust enough to reliably and reproducibly detect the presence multiple unique DNA (and/or RNA) sequences in a single test.

To remedy the above shortcomings, an advanced multiplex testing scheme is discussed herein. The technologies described herein can comprise computational multiplexing, which can be combined with physical multiplexing to form a testing scheme robust enough to reliably and reproducibly detect the presence of, for example, 20, 50, 100, 200, 500, 1,000 or greater numbers of unique DNA (and/or RNA) sequences within a sample in a single test. Specifically, the disclosed computational multiplexing technologies can be applied to, and/or combined with existing as well as newly developed and unique diagnostic assays, as well as physical multiplexing, for example, in a multichannel assaying of biological identities, and can be used for example, in applications for human diagnostics, including infectious disease uses and non-infectious disease uses, for example but not limited to, identification of rare genetic variants or identification of cancer mutations, pathogen surveillance, agriculture, veterinary diagnostics, and food safety applications. With the disclosed technologies, it may be possible to detect the presence of, for example, 20, 50, 100, 200, 500, 1,000 or greater numbers of unique DNA (and/or RNA) sequences by applying one or more of the disclosed embodiments, which may be suitable for human diagnostics, pathogen surveillance, agriculture, veterinary diagnostics, and food safety programs, among many other applications. An example embodiment of the disclosed computational multiplexing technologies can include a method for performing multiplexed diagnostic testing. In accordance with various embodiments, such method for performing multiplexed diagnostic testing may include providing a sample (e.g., from a subject) comprising a plurality of nucleic acid units to a plurality of channels of a multichannel device, followed by introducing a unique set of one or more probes and one or more reporter moieties into each of the plurality of channels. The method may include detecting a first indicator readout that creates a detectable signal (e.g., a visible signal such as from light emission, fluorescent, bioluminescence, or colorimetric reaction, an electrical signal, a radioactive signal) from one or more channels of the plurality of channels, wherein the first indicator readout is generated by at least one of the one or more reporter moieties as a result of interaction of a probe within the unique set with one of the nucleic acid units in the sample, in accordance with various embodiments. The method can further include generating a readout code for the sample based at least in part on the first indicator readout detected from the one or more channels of the plurality of channels, followed by matching the generated readout code against a plurality of readout code entries of a lookup table. In various embodiments, the plurality of readout code entries of the lookup table may represent one or more biological identities associated with the nucleic acid units. The method may then continue with generating a detection result of the plurality of biological identities for the sample based on the matched readout code.

There are key advantages offered by the disclosed computational multiplexing technologies. One advantage is that the disclosed multiplexing schemes (e.g., computational multiplexing or combined computational and physical multiplexing) can be applied to sequence-specific DNA (and RNA) detection technologies, which can offer rapid turnaround times and high multiplexing capabilities, and in certain circumstances, can be combined with low-cost instruments and consumables, and may provide access to, or work with, point-of-need devices. Moreover, the disclosed computational multiplexing technologies can be configured to detect the presence of large numbers of unique nucleic acid units in a sample, for example 20, 50, 100, 200, 500 or 1,000 units in, for example, a multichannel device, in accordance with various embodiments. In addition, the disclosed computational multiplexing technologies may be employed for many types of diagnostics. For example, the disclosed computational multiplexing technologies may be applied to screening for specific healthcare issues, for example, for detection of sepsis or respiratory disease, especially in neonates and seniors, as well as diagnosis for sexual transmitted infections (STIs), among many other health related applications. In various embodiments, the disclosed computational multiplexing technologies may be applied to proactive monitoring applications, such as for example, respiratory pathogen testing in schools, nursing homes, airports, embassies, military bases, mission-critical businesses, etc. The disclosed computational multiplexing technologies may be applied in high impact, high volume settings, such as to the generation of worldwide infectious disease maps.

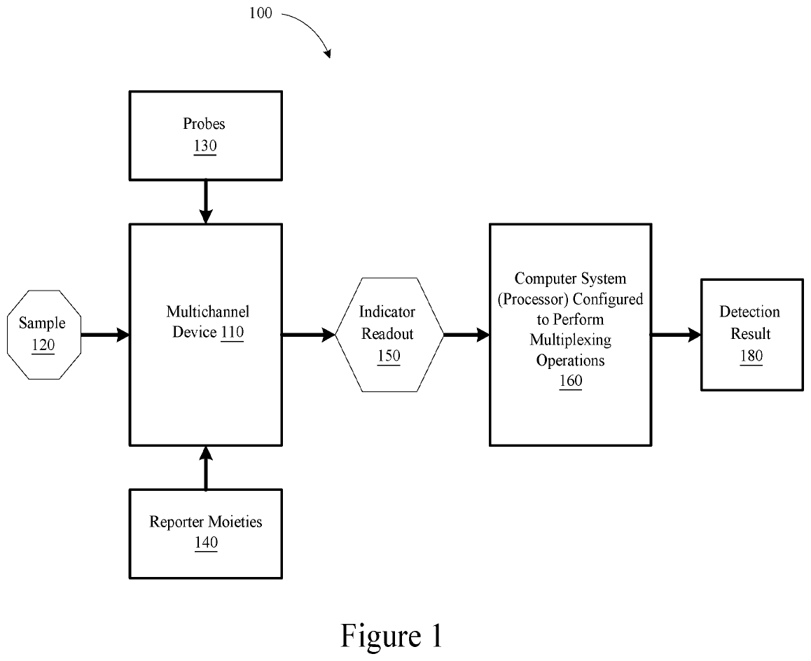

The disclosed computational multiplexing technologies are further illustrated and described with respect to FIGS. 1-13.

FIG. 1 illustrates an example of a system 100 for performing multiplexed diagnostic testing, in accordance with various embodiments. The system 100 illustrated and described herein encompasses the disclosed computational multiplexing technologies. As illustrated in FIG. 1, the system 100 includes a multichannel device 110, which is configured to receive a sample 120 comprising nucleic acid units for diagnostic testing. In various embodiments, the sample 120 can include, but is not limited to, a bodily substance selected from a group consisting of blood, saliva, urine, feces, and mucus. In various embodiments, the sample 120 includes nucleic acid units that comprise genomic information. In various embodiments, the nucleic acid units comprise DNA, RNA, or a combination thereof. In various embodiments, the nucleic acid units can comprise microbial genomic DNA, viral genomic information, or a combination thereof. In various embodiments, the nucleic acid units correspond to genomic nucleic acids of a subject. In some embodiments, each detected nucleic acid is from a different organism, such as a different microorganism, for example, a different bacterium, fungus and/or virus. In some embodiments, each detected nucleic acid is from the same organism, such as different genes, rDNA, coding or non-coding regions within an organism's genome. In some embodiments, detected nucleic acid are from different organisms and nucleic acid units from a same organism.

In various embodiments, the multichannel device 110 may include a plurality of channels, each of which is capable of receiving the sample 120 or a portion of the sample 120 for diagnostic testing. In various embodiments, the multichannel device 110 may include anywhere at least 2 channels and up to 100 channels. In some cases, the device includes 2, 4, 6, 8, 10, 12, 14, 16, 18, 20, 22, 24, 26, 28, 30, 40, 50, 60 or more channels. In some cases, the device includes between 2-10 channels, between 10-20 channels, between 20-50 channels. In accordance with various embodiments, each of the plurality of channels in the multichannel device 110 may be configured to receive a unique set of one or more probes 130 and one or more reporter moieties 140.

To describe the details of the unique set of one or more probes 130 and/or the one or more reporter moieties 140, and to further corroborate the disclosed computational multiplexing technologies, various embodiments of the technologies are described with CRISPR as an example process. Although CRISPR is used to illustrate the various embodiments disclosed herein, it is merely an example used to describe the capabilities of the disclosed computational multiplexing technologies. Thus, other processes, such as PCR, qPCR, etc., can also be used in conjunction with the disclosed computational multiplexing technologies.

In some exemplary embodiments, the disclosed computational multiplexing technologies are used with a nuclease assay, such as with a CRISPR system with a Cas nuclease, such as any of Cas9, Cas12, Cas12a, MAD4, MAD7, Cas13, or Cas14, and a plurality of guide RNAs compatible with such nuclease.

In various embodiments, the disclosed computational multiplexing technologies are used with a plurality of probes. In exemplary embodiments with a CRISPR system, the plurality of probes 130 are guide RNAs that are specifically chosen or designed to target for specific nucleic acid units, for example, DNA of pathogen(s). In various embodiments, the probes 130 (e.g., guide RNAs in exemplary embodiments) can be chosen such that each probe matches only a single target nucleic acid unit or a probe may match two or more nucleic acid units, such as DNA sequences common to a subset of pathogens. The type of probes will be determined by the type of assay employed for use with the disclosed computational multiplexing technologies. For example, if PCR is used with the disclosed computational multiplexing technologies, the probes 130 may include pairs of PCR primers where each pair amplifies a nucleic acid unit. In various embodiments, the probes 130 may include Taqman probes, hybridization probes, etc.

In various embodiments, the disclosed computational multiplexing technologies are used in conjunction with an assay that includes at least one reporter moiety 140. In some embodiments, the assay includes only one type of reporter moiety 140. In some embodiments, the assay includes more than one type of reporter moiety 140. The reporter moiety 140 is detectable, such as by visual, electrical, radioactive, or other means. In various embodiments, the reporter moiety 140 can be chosen based on its method of indication, e.g., a visible signal, a fluorescent signal, a bioluminescent signal, a light-emitting signal, a radioactive emission, an electrical signal, and any combination thereof. For example, the reporter moiety 140 may be an enzyme, such as luciferase. In various embodiments, the reporter moiety 140 can be chosen for its method of indication, e.g., bioluminescent or colorimetric emission. In some cases, the reporter moiety 140 may be soluble in solution; in some cases, the reporter moiety 140 may be tethered to a solid substrate, such as a surface or a bead.

As illustrated in FIG. 1, the multichannel device 110 is configured to detect an indicator readout 150 (e.g., a first indicator) from one or more channels (e.g., a first set of channels) of the plurality of channels of the multichannel device 110. In various embodiments, the indicator readout 150 is generated by interaction of one or more of the probes 130 within the unique set of probes in the channel with one of the nucleic acid units in the sample 120. In various embodiments, the type of indicator readout 150 is determined by the selected reporter moiety 140 and can include, for example, light emission, fluorescent, bioluminescence, or colorimetric signal, etc. In various embodiments, the indicator readout 150 can be selected from a group consisting of a visible signal, a fluorescent signal, a bioluminescent signal, a light-emitting signal, a radioactive emission, an electrical signal, and any combination thereof.

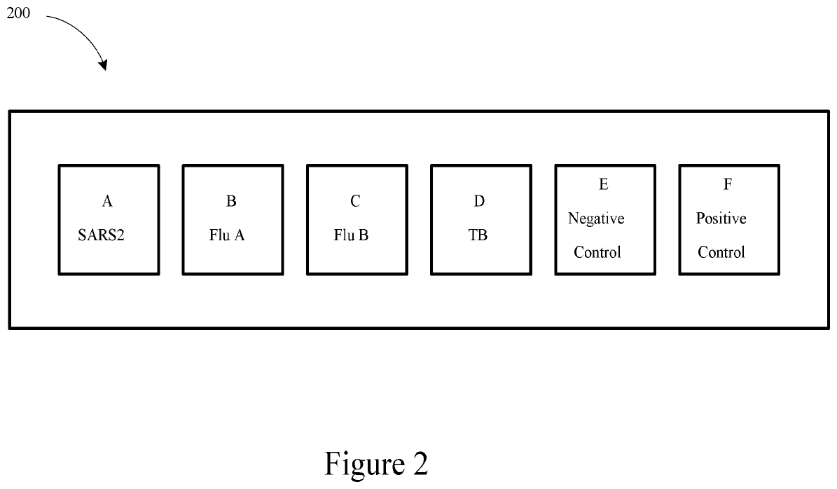

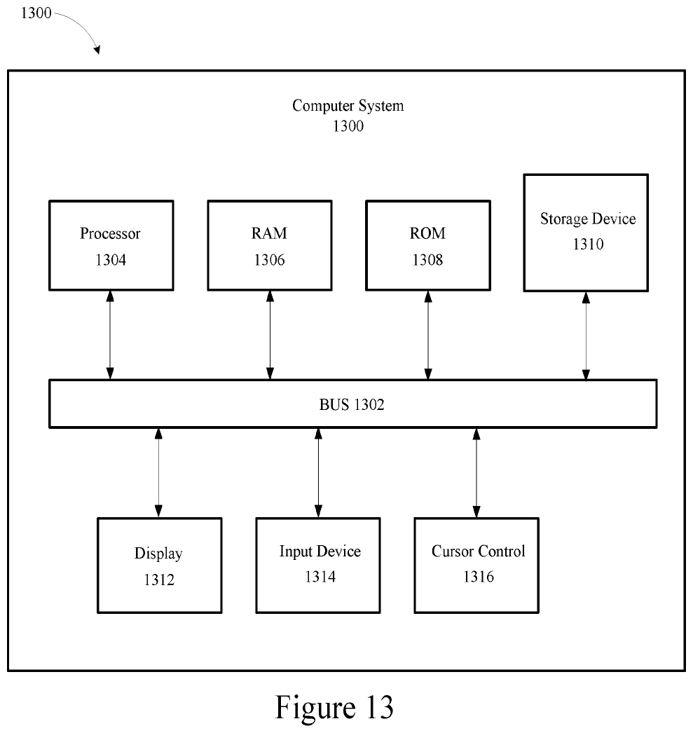

As further illustrated in FIG. 1, the indicator readout 150 can be fed into a computer system 160 that includes a processor that is configured for performing multiplexing operations. The computer system 160 can be any computing system, device, or platform that can perform computing operations, such as the computer system 1300 as described with respect to FIG. 13. Once the indicator readout 150 is fed into the computer system 160, it can be matched against a plurality of readout code entries of a lookup table. The lookup table can be loaded in the computer system 160 or can be stored in the cloud or on a network server that is communicatively connected to the computer system 160. In various embodiments, the plurality of readout code entries of the lookup table represents a plurality of biological identities associated with nucleic acid units that may be present in a sample. Once a match is found, the computer system 160 generates a detection result 180 for the sample based on the matched readout code. Additional details are further described with respect to FIGS. 2-11.

FIG. 2 illustrates an example testing scheme 200 for performing multichannel diagnostic testing without the computational multiplexing described herein. As illustrated in FIG. 2, the testing scheme 200 uses a multi-channeling approach that includes 6 channels (A-F) of a multichannel device (such as the multichannel device 110 of FIG. 1), where each of the first 4 channels (A-D) are designated to test a specific pathogen (or biological identity). For example, channel A is designated to test SARS 2 pathogen using one or more probes for SARS 2, whereas channel B includes probe(s) for Flu A, channel C includes probe(s) for Flu B, and channel D includes probe(s) for TB. As for channels E and F, they are used as negative control and positive controls, respectively. The detection of the specific pathogens (SARS 2, Flu A, Flu B, and TB) occurs when a channel holding the specific guide lights up to indicate the presence of the pathogen in that channel. If specific channel fails, the failure can lead to an incorrect result for the entire test.

To remedy a single channel failure from causing an incorrect for the entire test, a specifically designed algorithm can be designed in conjunction with computational multiplexing, as disclosed herein.

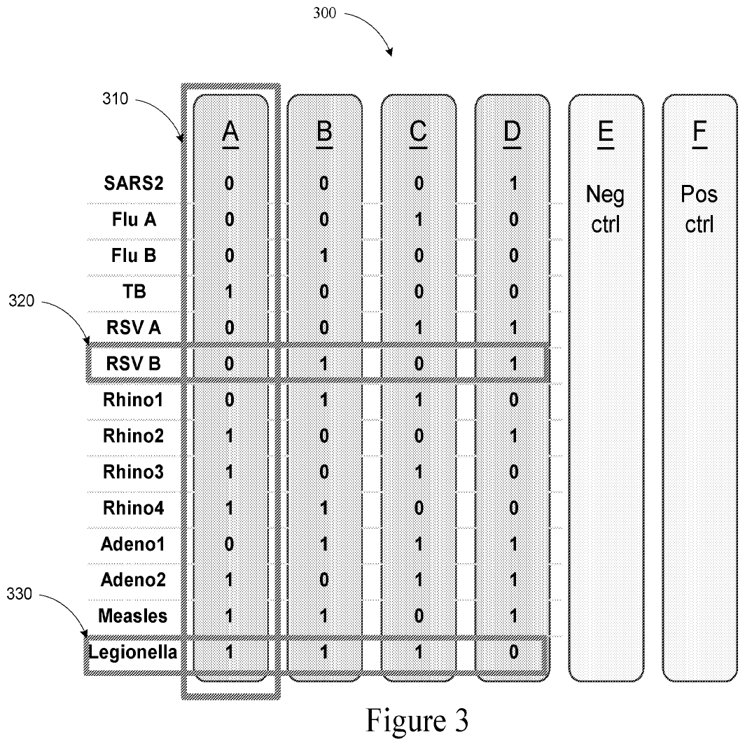

FIG. 3 illustrates an example testing scheme 300 for performing multiplexed diagnostic testing, in accordance with various embodiments. As illustrated in FIG. 3, the testing scheme 300 uses a multi-channeling approach, here exemplified with 6 channels (A-F) of a multichannel device (such as the multichannel device 110 of FIG. 1), where, instead of the scheme as described in FIG. 2, the first 4 channels (A-D) of FIG. 3 are combined to perform a computational multiplexing. As for channels E and F, they are used as negative control and positive controls, respectively. In the approach illustrated in FIG. 3, a binary method of the testing scheme 300 is used to extract the detection result. As illustrated in FIG. 3, each of the 4 channels A-D is loaded with a selection of probes. For example, channel A is loaded with probes to TB, Rhino 2, Rhino 3, Rhino 4, Adeno 2, measles, and legionella, as indicated by box 310, channel B is loaded with Flu B, RSV B, Rhino 1, Rhino 4, Adeno 1, measles, and legionella, and so on and so forth for channel C and channel D. By using a binary code of “0” and “1”, the 4-channel multi-channeling approach can be multiplexed computationally to detect up to 14 pathogens, as illustrated in FIG. 3. For example, if the 4-channel (A-D) reads “0101”, the generated detection result would indicate a presence of the RSV B pathogen, as indicated by box 320. If the 4-channel (A-D) reads “1110”, the generated detection result would indicate a presence of the legionella pathogen, as indicated by box 330.

As for data analysis based on the binary method, the detection result can be generated in a form of a binary code that can be compared to a look up table to determine which of the pathogens the binary code corresponds to. Based on the number of “N” channels, there is a maximum 2ⁿ binary codes that are available to be used in the look up table. For example, if a 10-channel multichannel device is used, then there are 2¹⁰=1,024 uniquely available binary codes, and there are 2²⁰=1,048,576 uniquely available binary codes if 20 channels of the multichannel device are used. Thus, the concept of computational multiplexing illustrated in FIG. 3 can be scaled up to any number of channels as desired.

In some cases, there may be a co-infection or a single channel failure that will lead to an incorrect result. For example, if co-infection occurs with Rhino 4 and legionella, with Rhino 4's binary code being 1100 and legionella's binary code 1110, in the described exemplary embodiment, it would lead to an incorrect result. If a particular channel is not functioning, i.e., a single channel failure, the channel will read “0” at all times, limiting the results to pathogens for which the binary code of the failed channel indicates “0”.

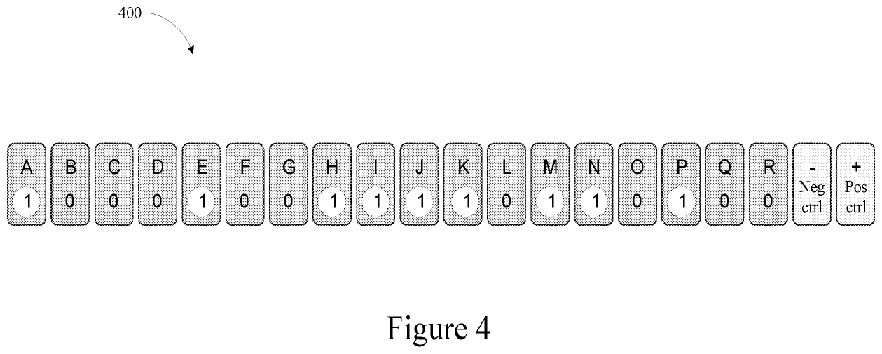

FIG. 4 illustrates an example testing scheme 400 for performing multiplexed diagnostic testing, in accordance with various embodiments. As illustrated in FIG. 4, the testing scheme 400 uses a multi-channeling approach, in this exemplary figure, it includes 20 channels, including channels A-R, and channels with negative and positive controls, of a multichannel device (such as the multichannel device 110 of FIG. 1). Expanding on the binary method described with respect to FIG. 3, two additional methods of binary coding are contemplated to reduce coinfection problem and make testing scheme 400 more robust to errors. The first approach is Hamming codes, which can be used in error correction, and the second approach is a binomial coefficient (nCk, “n choose k”) method. Derived from the combinatoric operation of the same name, with this approach, a code for each diagnostic target is a unique selection of k channels out of n, or, phrased differently, each code is a unique binary digit n-bits long with exactly k bits set to 1. Since all codes have exactly k bits set to 1, no code is a strict subset or superset of another code, which eliminates the possibility of overlaps being “hidden” by yielding one single valid code. When more than one diagnostic target is present, more than k channels are expected to display a signal. Furthermore, when a certain number of 1's become flipped to 0's, it also will not yield another valid code since no code with fewer than k 1s is a valid code. Another ancillary benefit is that the presence of errors (specifically omissions and overlaps) can be easily confirmed; for example, if the reporter is a colorometric or luminescent detection system, the presence of more than k colored or luminescent channels in the multichannel device indicate an error of some kind.

In the most basic case, n can be as high as the number of physical channels in a multichannel device. However, it may be desirable to reserve one or more channels for positive/negative controls, in which case n may be 1 or more fewer than the number of physical channels. In other situations, it may be desirable to split the physical channels further into separate distinct zones.

There are a number of considerations for the value of k. To maximize “sparseness” or the edit distance between codes, a value of k that is n/2 yields the largest number of possible permutations. However, using a lower value for k can allow overlaps to be correctly disambiguated more often.

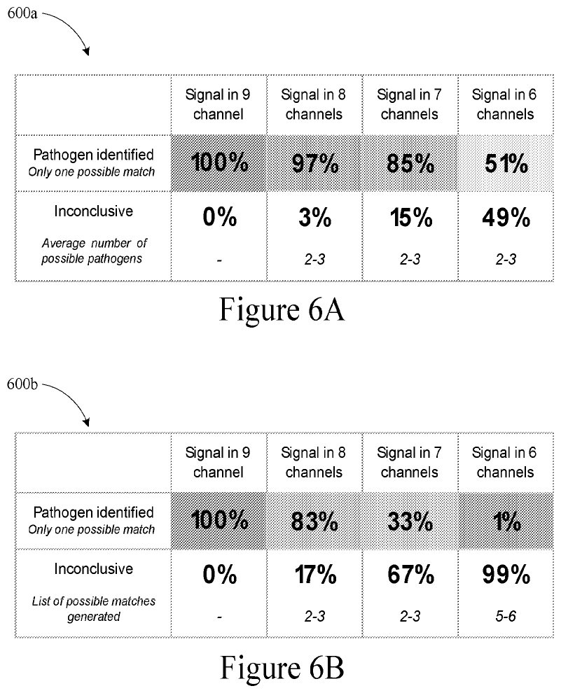

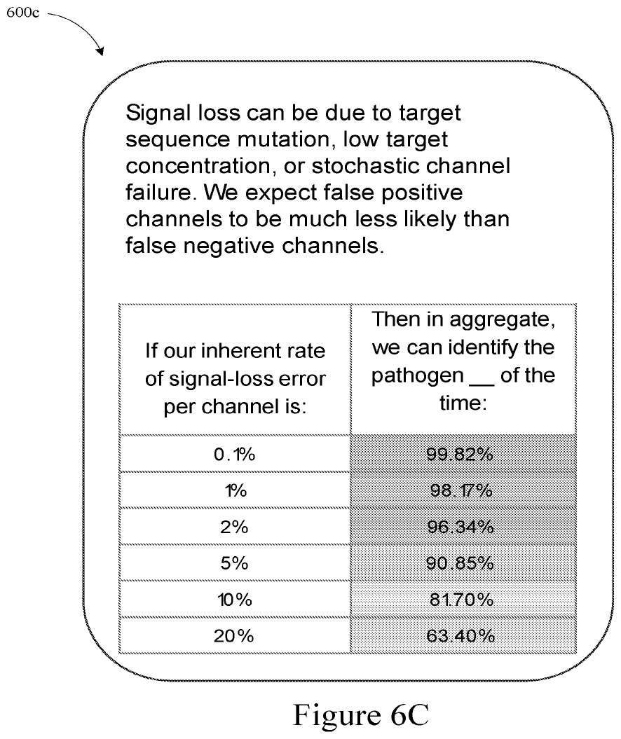

For any given n and k, the maximum number of targets that can uniquely be encoded can be calculated using the “n choose k” operation (or n!/k!(n-k)!). However, using all available codes will diminish the ability to correct for omissions. For example, if there is a single omission (i.e., k-1 channels luminesce), if all possible codes are used, there will be n-k codes that share those k-1 channels. In general, the number of targets to encode becomes a trade-off between the number of targets that can be identified in the ideal case (where there are no errors) and the ability to correct for errors. For example, with n=18 and k=9, it is possible to support 1000 targets (codes) while retaining the ability to correct 1 omission (i.e., one bit flip of a “1” to a “0”) 97% of the time, to correct 2 omissions 85% of the time, and to correct 3 omissions 51% of the time (FIG. 6A).

There are a few different ways that codes can be selected that meet the n choose k constraint for use with the multiplexed diagnostic testing methods described herein.

Random Selection—The simplest and most naive approach is to randomly choose k channels while ensuring uniqueness. With this approach, it is not guaranteed that the codes are evenly distributed, and it is possible that some codes that are selected are close together.

Hamming Codes—One approach with predictable properties is to select Hamming Codes with length n that have k 1's set. This has the advantage of guaranteeing error correction abilities and edit distances of chosen codes, though the maximum number of possible codes is limited to those that satisfy such constraints.

Most Distant—Another approach is to choose codes such that for each new code chosen, the most distant code is selected. In one such implementation, the first code may be selected randomly, but then each successive code is chosen by first calculating the distance between all possible candidate codes and all previously chosen codes, then choosing the candidate code that has the highest distance to previously chosen codes. Here, the definition of “distance” could yield different results. One approach is to take the maximum minimum distance. That is, for each candidate code, the minimum distance to previously chosen codes is calculated (i.e., if a candidate code has distance of 1 to even one of the previously chosen codes, the “distance” is 1), and the candidate code with the largest such minimum distance is chosen. Distance between two codes A and B corresponds to how many “1's” are changed to “0's” and how many “0's” are changed to “1's”.

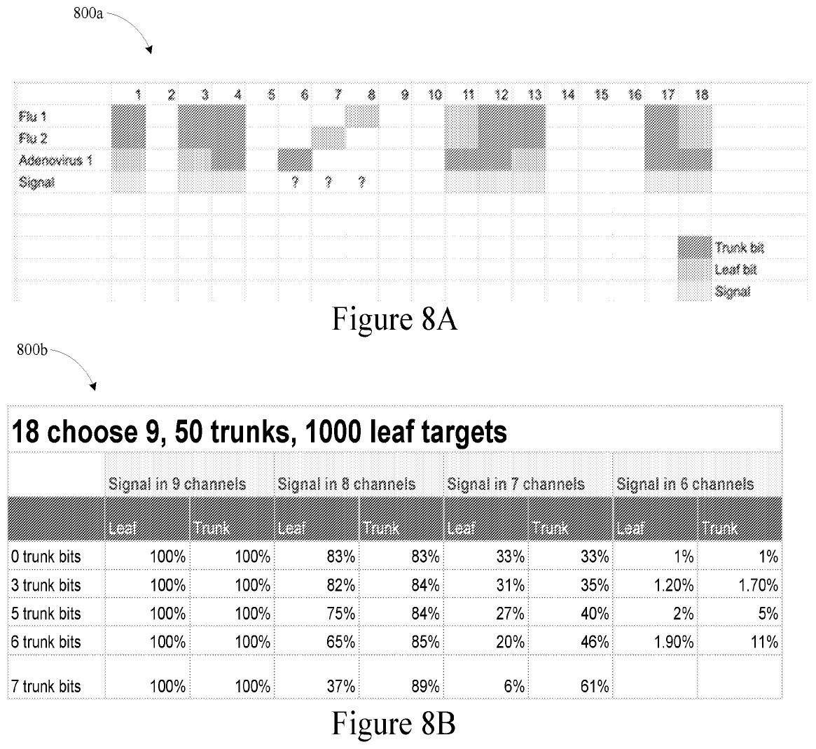

Variable Distance—In some cases, it may be desirable to create variability in the sparseness of codes. For example, it may be desirable for a subset of codes to have an edit distance of 2 or more to other codes, thereby allowing those codes to tolerate 2 or more errors, while other codes may have distance of 1 or less and be less tolerant to errors. One method of creating such variability is discussed in the “trunk and leaf” section below (with respect to FIGS. 8A and 8B), while a variation of the “Most Distant” algorithm described above could also be used.

As illustrated in the exemplary embodiment in FIG. 4, channels A-R gives 18 channels, and applying “18 choose 9” method, there are C(18,9)=48,620 binary codes that use exactly 9 “1”s, out of 2¹⁸=262,144 total available codes. For example, to detect 150 biological identities, such as 150 pathogens, 150 binary codes or 0.3% of 48,620 binary codes that use exactly 9 “1”s can be used. The remaining 99.7% (i.e., 48,470) do not represent any biological identities for detection. To detect 1000 biological identities, 1000 codes or 2% of 48,620 binary codes can be used, where the remaining 98% do not represent any biological identities.

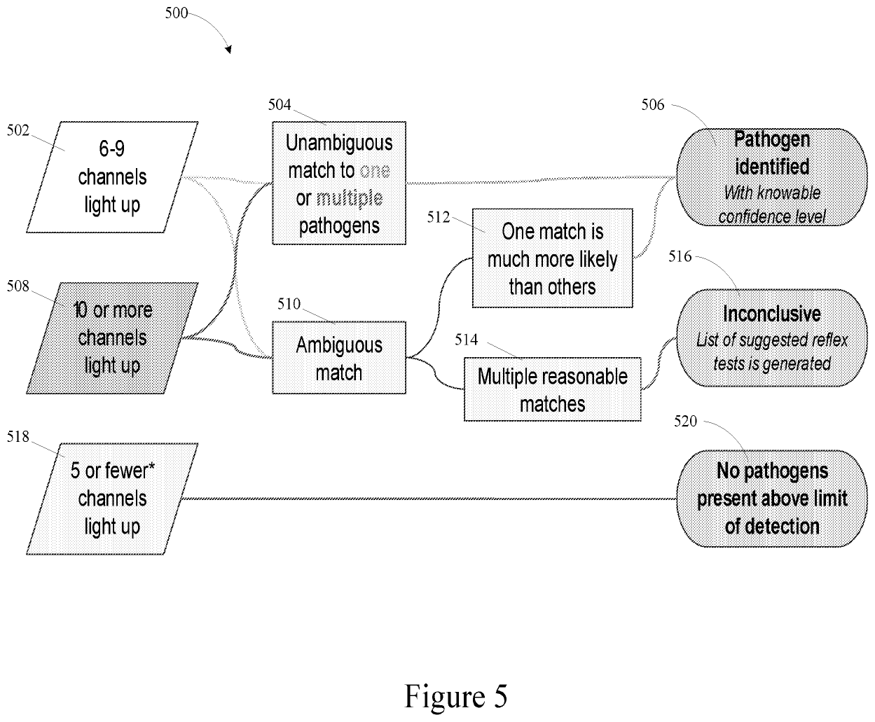

FIG. 5 illustrates an example scenario 500 resulting from the testing scheme 400 for multiplexed diagnostic testing, in accordance with various embodiments. As illustrated in FIGS. 4 and 5, if the “18 choose 9” method is used, any given pattern of “0” and “1” channels conclude in one of three possible results: a positive diagnosis, a suggestion for follow-up tests, or a negative diagnosis. For example, if 6-9 channels are indicated as “1” (e.g., light up) as shown in box 502, the result leads to unambiguous match to one or multiple biological identities (e.g., pathogens) as shown in box 504, and results in such biological identities being identified with knowable confidence level as shown in box 506. If 10 or more channels are indicated as “1” (e.g., light up) as shown in box 508, the result either leads to unambiguous match to one or multiple biological identities as shown in box 504, or ambiguous match as shown in box 510, which can further filter to “one match is much more likely than others” as shown in box 512, or “multiple reasonable matches” as shown in box 514. If the one match is much more likely than others as shown in box 512, the biological identities (e.g., one or more pathogens) can be identified with knowledgeable confidence level as shown in box 506. If multiple reasonable matches as shown in box 514, the result leads to “inconclusive”, with indication of a list of suggested reflex tests to be generated as shown in box 516. If 5 of fewer channels light up as shown in box 518, it can result with no biological identities being detected or indicated as no biological identities (e.g., pathogens) present above detection limit of the multichannel device as shown in box 520. In various embodiments, if 5 or fewer channels light up or indicated as “1”, it can be defined as negative in an arbitrary cutoff, which may be further adjusted later based on additional/modeling/prevalence data, etc.

FIGS. 6A, 6B, and 6C illustrate an example scenario 600a, 600b, and 600c, respectively, of data interpretations, in accordance with various embodiments, such as for detecting of one or more pathogens in a sample. FIG. 6A illustrates the scenario 600a, in which 150 pathogens are tested in a 20-channel multichannel device with randomly selected codes, in accordance with various embodiments. When a single pathogen (biological identity) is present, a pathogen is identified with one possible match with 100% certainty and inconclusive/uncertainty percentage of 0% when there are signals “1” in 9 channels. If there are “1”s in 8 channels, the percentage of a pathogen being identified with one possible match goes down to 97%, with 3% inconclusive/uncertainty percentage, with 2-3 average number of possible pathogens. If there are “1”s in 7 channels, the percentage of a pathogen being identified with one possible match goes down to 85%, with 15% inconclusive/uncertainty percentage, with 2-3 average number of possible pathogens. If there are “1”s in 6 channels, the percentage of a pathogen being identified with one possible match goes down to 51%, with 49% inconclusive/uncertainty percentage, with 2-3 average number of possible pathogens.

FIG. 6B illustrates the scenario 600b, in which 1000 pathogens are tested in a 20-channel multichannel device, in accordance with various embodiments. When a single pathogen (biological identity) is present, a pathogen is identified with one possible match with 100% certainty and inconclusive/uncertainty percentage of 0% when there are signals “1” in 9 channels. If there are “1”s in 8 channels, the percentage of a pathogen being identified with one possible match goes down to 83%, with 17% inconclusive/uncertainty percentage, with 2-3 in a list of possible matches generated. If there are “1”s in 7 channels, the percentage of a pathogen being identified with one possible match goes down to 33%, with 67% inconclusive/uncertainty percentage, with 2-3 in a list of possible matches generated. If there are “1”s in 6 channels, the percentage of a pathogen being identified with one possible match goes down to 1%, with 99% inconclusive/uncertainty percentage, with 5-6 in a list of possible matches generated.

FIG. 6C illustrates the scenario 600c due to signal loss errors, in accordance with various embodiments. As discussed herein, a signal loss can be due to, for example, target sequence mutation, low target concentration, or stochastic channel failure, to name a few. In various embodiments, the false positive channels are expected to be much less likely than false negative channels. FIG. 6C illustrates various inherent rates of signal loss error per channel for which a pathogen can be identified various percentage of the time. For example, 0.1% signal loss error per channel, 99.82% of the time a pathogen can be identified. If signal loss error per channel goes to 5%, for example, a pathogen can be identified 90.85% of the time. For 20% of signal loss error per channel, then the percentage goes down to 63.4% of the time.

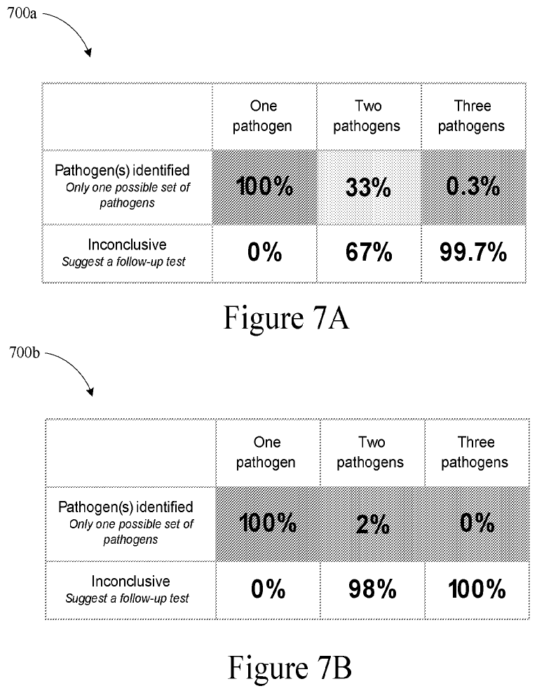

FIGS. 7A and 7B illustrate an example scenario 700a and 700b, respectively, of data interpretations, in accordance with various embodiments. FIG. 7A illustrates the scenario 700a, in which 150 biological identities (in the example, 150 pathogens) are tested in a 20-channel multichannel device, in accordance with various embodiments. When there is signal of “1” in 9 channels, there is 100% certainty that a single pathogen (biological identity) is identified and inconclusive/uncertainty percentage of 0%. For the signal of “1” in 9 channels, the likelihood of detecting two pathogens goes to 33% certainty of the pathogens being identified or only one possible set of pathogens, and inconclusive/uncertainty percentage of 67% to suggest a follow up test may be needed. For the same signal of “1” in 9 channels, the likelihood of detecting three pathogens goes further down to 0.3% certainty of the pathogens being identified or only one possible set of pathogens, and inconclusive/uncertainty percentage of 99.7% to suggest a follow up test may be needed.

FIG. 7B illustrates the scenario 700b, in which 1000 pathogens are tested in a 20-channel multichannel device, in accordance with various embodiments. When there is signal of “1” in 9 channels, there is 100% certainty that a single pathogen (biological identity) is identified and inconclusive/uncertainty percentage of 0%. For the signal of “1” in 9 channels, the likelihood of detecting two pathogens goes to 2% certainty of the pathogens being identified or only one possible set of pathogens, and inconclusive/uncertainty percentage of 98% to suggest a follow up test may be needed. For the same signal of “1” in 9 channels, the likelihood of detecting three pathogens goes further down to 0% certainty of the pathogens being identified or only one possible set of pathogens, and inconclusive/uncertainty percentage of 100% to suggest a follow up test is needed.

In various embodiments, multiplexing with the disclosed nCk method is most effective in a population where coinfections are unlikely. However, the nCk method may not increase the risk of giving an incorrect result with a coinfection is present; rather an inconclusive result may likely suggest a follow up test.

FIGS. 8A and 8B illustrates tables 800a and 800b, respectively, from the results of an example coding scheme used in performing for multiplexed diagnostic testing, in accordance with various embodiments. Tables 800a and 800b show various values generated from a coding scheme that uses n choose k method with another coding scheme, a trunk and leaf approach. The “trunk and leaf” coding scheme can represent a 2-level hierarchy, wherein a trunk represents some higher-level grouping of targets, while the leaves represent more specific targets within such grouping. For example, a trunk can be “Influenza”, while the leaves underneath may each represent specific variants of the flu (H5N1, H1N1, etc.). This is accomplished by designating certain bits of the binary codes to represent the trunk (“trunk bits”), while the remaining bits of the code represents the leaves (“leaf bits”). For example, all codes in a given trunk may have 1s in bits 1 through 3, while the remaining 15 bits (if there are 18 total channels/bits) would be used to encode the leaf. In some embodiments, there could be more than two layers in the hierarchy, e.g., trunk-branch-leaf, or trunk-branch1-branch2-leaf, etc.

Furthermore, the trunk bits can be configured to be more resilient to bit-flipping. This can be accomplished by having a proportionally larger amount of probe in each of the channels of the multichannel device, such as multichannel device 110 of FIG. 1, by selecting probes with higher efficiency, and/or by targeting multiple target sequences to increase resilience to mutations. In various embodiments, Trunk bits are not “reserved”, in that if Trunk 1 uses bits 1, 2, and 3, no other trunk would use that exact set of bits as their trunk bits, but other trunks and other leaves may use bits 1, 2, and 3 (e.g., 1, 2, and 4 could be a valid trunk bit). A coding scheme where trunk bits are non-overlapping (i.e., if Trunk 1 uses bits 1-3, then those bits cannot be used as trunk bits) is possible. However, this can limit the number of possible trunks (e.g., with 18 channels and 3 trunk bits, there can only be 6 trunks total if their bits can't overlap).

As shown in Table 800a of FIG. 8A, the bit patterns for 3 different targets are shown, with green indicating the positions of 1s (i.e., channels that should luminesce), and dark green representing trunk bits while light green represent leaf bits (in this example, there are 6 trunk bits and 3 leaf bits). The yellow squares represent a hypothetical result with 8 luminescent channels. The question marks represent the channels that may have dropped out. With Trunk and Leaf, because Trunk bits are designed to have lower drop-out rates, it is most likely that either channel 7 or 8 that drops out, and that therefore the sample is most likely positive for Flu 1 or Flu 2. More significantly, the sample can be inferred to be most likely positive for Flu and not Adenovirus, even though it is not possible to determine the particular strain of flu.

Table 800b of FIG. 8B illustrates various values of 18 choose 9 method combined with 50 trunks and 1000 leaf targets. The numbers are improved by considering the phylogenetic relationships between pathogens and assigning channels as “trunks” and “leaves”. However, since each channel that's reassigned from leaf to trunk means only half as many codes remain accessible, this method results in a tradeoff where trunk identification improves slightly but leaf identification falls significantly.

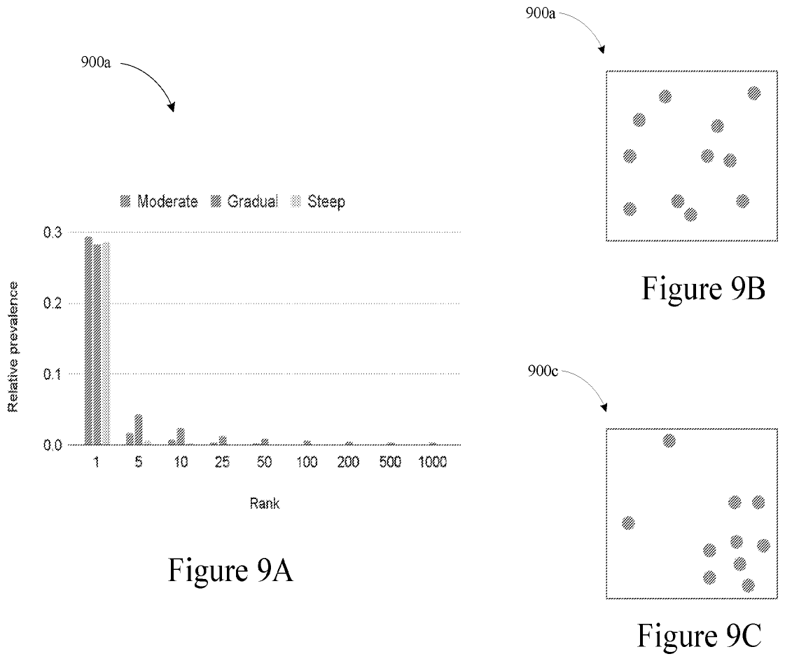

FIGS. 9A, 9B, and 9C illustrate how prevalence rates affect computational multiplexing results, in accordance with various embodiments. FIG. 9A shows a graph 900a illustrating three modeled prevalence distributions. For example, this could represent different pathogens, of which some are more prevalent in a given location and season and others less prevalent; or different genetic markers, of which some are more prevalent in a given population and others less prevalent. FIG. 9B illustrates plot 900b that is randomly assigned codes and FIG. 9C illustrates plot 900c that has some bits more isolated and others less isolated. Assigning higher-prevalence diagnostic targets to codes that are more isolated can be beneficial to the computational multiplexing to yield more conclusive results, meaning less likely to yield inconclusive results.

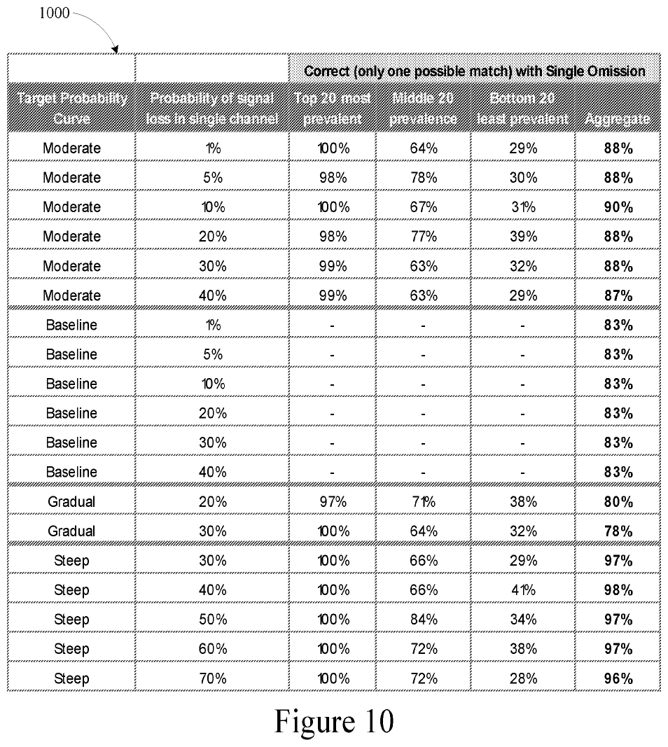

FIG. 10 illustrates a table 1000 displaying results when prevalence rates are taken into account to improving test results, in accordance with various embodiments. Table 1000 is an example generated for 1000 pathogens using 18 channels with signal “1” in 9 channels. As illustrated, even with the moderately steep prevalence curve, the success rate and robustness to signal loss is significantly improved when prevalence is considered. Thus, the pathogen prevalence data can help significantly in improving diagnostic results. However, in some cases, low sensitivity for the less prevalent biological identities, such as for a less prevalent pathogen, risks generating low positive predictive values (PPV) for those pathogens.

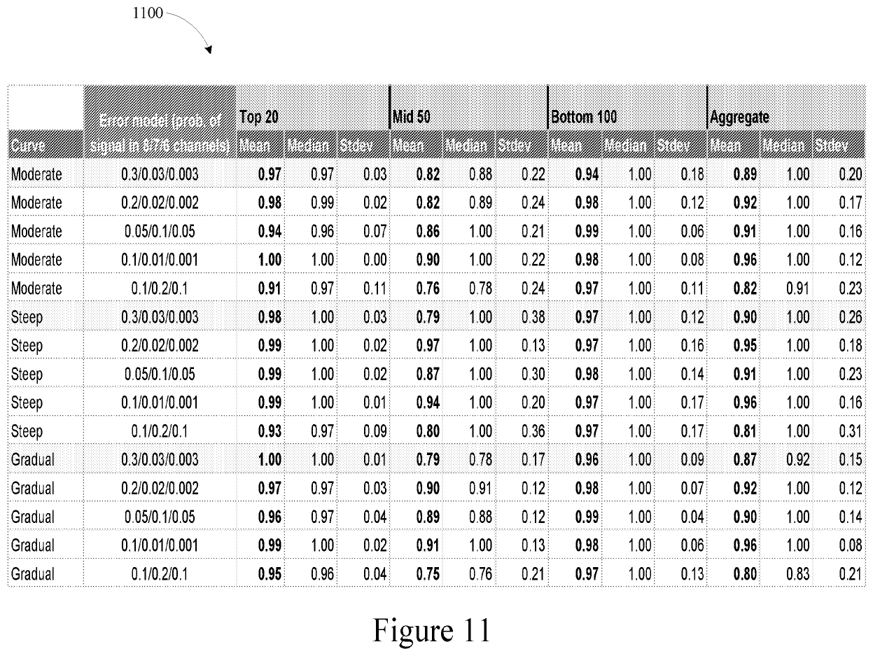

FIG. 11 illustrates a table 1100 displaying positive predictive values given various prevalence rate curves and error models, in accordance with various embodiments. Table 1100 is generated for 1000 pathogens using a 20-channel multichannel device with positive predictive values (PPV). In some cases, because both false positives and true positives are lower for the low prevalence pathogens, the PPV remains in a reasonable range.

Taking into account various aspects of the disclosed computational multiplexed diagnostic testing, a general method of performing a multiplexed diagnostic testing is described as follows.

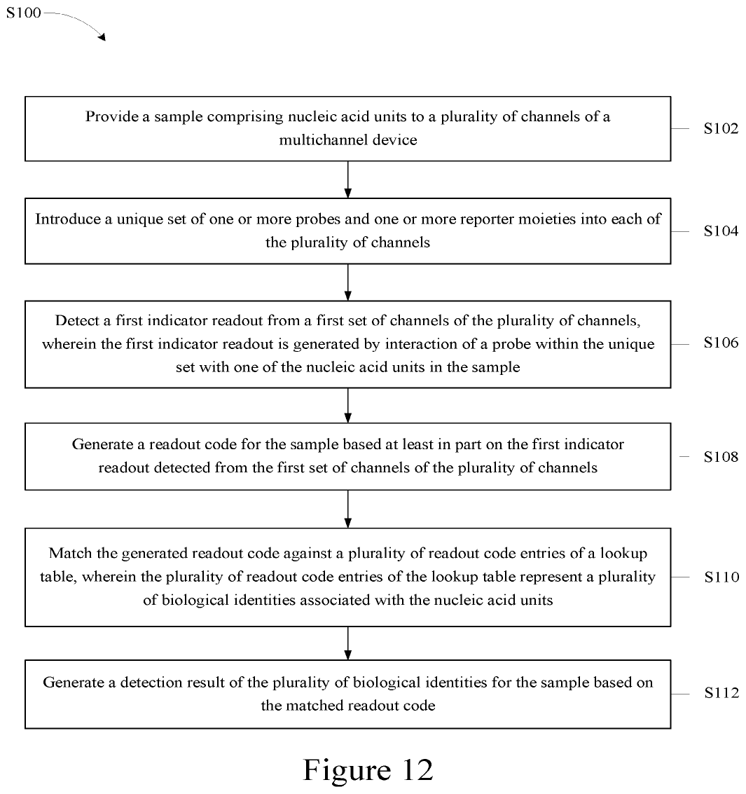

FIG. 12 illustrates an example method S100 for performing multiplexed diagnostic testing, in accordance with various embodiments. In accordance with various embodiments, the method S100 can be implemented using the system 100 as described with respect to FIG. 1. As illustrated in FIG. 12, the method S100 includes, at step S102, providing a sample comprising nucleic acid units to a plurality of channels of a multichannel device; at step S104, introducing a unique set of one or more probes and one or more reporter moieties into each of the plurality of channels; at step S106, detecting a first indicator readout from a first set of channels of the plurality of channels, wherein the first indicator readout is generated by at least one of the one or more reporter moieties as a result of interaction of a probe within the unique set with one of the nucleic acid units in the sample; at step S108, generating a readout code for the sample based at least in part on the first indicator readout detected from the first set of channels of the plurality of channels; at step S110, matching the generated readout code against a plurality of readout code entries of a lookup table, wherein the plurality of readout code entries of the lookup table represent a plurality of biological identities associated with the nucleic acid units; and at step S112, generating a detection result of the multiplexed diagnostic testing for the sample based on the matched readout code.

In various embodiments, matching the generated readout code against a plurality of readout code entries of a lookup table can include, for example, a complete match, an exact match, a closest possible match, or a substantial match. In various embodiments, a closest possible match can be determined using an inference that can be made upon checking with the available readout code entries in the lookup table. For example, upon checking the lookup table and an exact code isn't present, an inference can be made with the closest possible match to indicate that the generated readout code has been compared against a plurality of readout code entries of a lookup table. The inference may take into account other data, such as prevalence rates, coinfection rates, or other information about the subject. In various embodiments, if there are multiple possible matches for a given generated readout code, but one of them is not consistent with the clinical sample type, with the demographics of the subject/patient etc., then that readout code entry may be eliminated/downgraded in likeliness from the list of possible matches.

In various embodiments, the plurality of channels each include the same reporter moiety. In various embodiments, the nucleic acid units include genomic information. In various embodiments, the nucleic acid units include DNA, RNA, or a combination thereof. In various embodiments, the plurality of biological identities are a plurality of pathogens and the nucleic acid units correspond to nucleic acids of the plurality of pathogens. In various embodiments, the nucleic acid units include microbial genomic DNA, viral genomic information, or a combination thereof. In various embodiments, the nucleic acid units correspond to genomic nucleic acids of a subject. In various embodiments, the plurality of biological identities include genetic disease markers. In various embodiments, the plurality of biological identities include cancer-associated markers. In various embodiments, the unique set of one or more probes are guide RNAs and each of the plurality of channels further includes a CRISPR type nuclease. In various embodiments, the CRISPR type nuclease is selected from a group consisting of Cas9, Cas12, Cas12a, MAD4, MAD7, Cas13, and Cas14. In various embodiments, one probe (such as a guide RNA) or one pair of probes (such as a pair of PCR primers) is engineered for interacting with one nucleic acid unit. In various embodiments, one probe or one pair of probes is engineered for interacting with more than one nucleic acid unit, such as with a group of related nucleic acid units (e.g., nucleic acid units from related pathogens). In various embodiments, the first indicator readout is selected from a group consisting of a visible signal, a fluorescent signal, a bioluminescent signal, a light-emitting signal, a radioactive emission, an electrical signal, and any combination thereof.

In various embodiments, the generating the readout code includes assigning one of two symbols of a bit for the first set of channels to generate a binary readout code. In various embodiments, the method further includes failing to detect a second indicator readout from a second set of channels of the plurality of channels different from the first set of channels, wherein the generating the readout code includes assigning the other symbol of the two symbols of the bit for the second set of channels to generate the binary readout code.

In various embodiments, each readout code entry of the plurality of readout code entries corresponds to one of the 2ⁿ n-bit binary codes where n is a number of the plurality of channels; and the matching includes comparing the binary readout code to the one of the 2ⁿ n-bit binary codes. In various embodiments, the detection result indicates that the sample contains one of the plurality of biological identities represented by the one of the 2ⁿ n-bit binary codes when the comparison indicates that the binary readout code matches the one of the 2ⁿ n-bit binary codes.

In various embodiments, each readout code entry of the plurality of readout code entries corresponds to one of binomial coefficient (n;k) number of binary codes where n is a number of the plurality of channels and k bits of each binary code share a same symbol different from symbol of any other bit of the binary code; and the matching includes comparing the binary readout code to the one of binomial coefficient (n;k) number of binary codes. In various embodiments, the detection result indicates that the sample contains one of the plurality of biological identities represented by the one of binomial coefficient (n;k) number of binary codes when the comparison indicates that the binary readout code matches the one of binomial coefficient (n;k) number of binary codes. In various embodiments, k is equal to a natural number ranging from 1 to n/2. In various embodiments, the method further includes assigning high-prevalence biological identities to certain binary codes with higher distance away from other used codes, and/or assigning lower-prevalence biological identities to binary codes with lower distance away from other codes.

In various embodiments, each readout code entry of the plurality of readout code entries belongs to a group of binary codes where trunk bits of each of the grouped binary codes located at same binary positions share same symbol; and the matching includes performing a first comparison of the trunk bits of the binary readout code located at the same binary positions to the trunk bits of any of the grouped binary codes. In various embodiments, the first comparison indicates that the binary readout code belongs to the group of binary codes; the binary readout code entry corresponds to one of the grouped binary codes of the group of binary codes; and the matching further includes performing a second comparison of bits of the binary readout code other than the k bits of the binary readout code to bits of one of the grouped binary codes other than the k bits of the one of the grouped binary codes. In various embodiments, the diagnostic test result indicates that the sample contains one of the plurality of biological identities represented by the one of the grouped binary codes when the second comparison indicates that the bits of the binary readout code other than the k bits of the binary readout code match the bits of one of the grouped binary codes other than the k bits of the one of the grouped binary codes.

In various embodiments, the sample containing one or more nucleic acid units includes a bodily substance selected from a group consisting of blood, saliva, urine, feces, and mucus. In various embodiments, the plurality of biological identities include pathogens from bacteria, fungi and/or viruses, such as from one or more of a rhinovirus, a coronavirus, an influenza virus, a tuberculosis pathogen, a respiratory syncytial virus (RSV), an adenovirus, a measles virus, a legionella bacterium, SARS-COV-2 virus, type A influenza virus and/or a type B influenza virus.

In various embodiments, the method further includes providing the sample to a first control channel and a second control channel of the multichannel device, wherein the first control channel is capable of identifying a first biological identity in the sample and wherein the second control channel is incapable of identifying the first biological identity in the sample. In various embodiments, the method further includes validating the detection result when the first control channel identifies the first biological identity in the sample and/or the second control channel fails to identify the first biological identity in the sample. In various embodiments, the method further includes invalidating the detection result when the first control channel fails to identify the first biological identity in the sample and/or the second control channel identifies the first biological identity in the sample.

In various embodiments, each of the plurality of channels includes the unique set of one or more probes that are configured to detect at least 1, 5, 10, 20, 25 or more than 25 nucleic acid units. In various embodiments, the plurality of channels is configured to detect at least 1, 2, 5, 10, 20, 30, 40, 50, 100, 200, 500, 1000 biological identities. In various embodiments, the detection result of the multiplexed diagnostic testing for the sample includes an identification of one biological identity from the plurality of biological identities listed in the plurality of readout code entries of the lookup table.

In various embodiments, the method further includes applying a prevalence value of specific biological identities to the lookup table. In various embodiments, the specific biological identities include pathogens, and the method further includes applying a rate of coinfections of specific biological identities to the lookup table.

FIG. 13 is a block diagram illustrating a computer system 1300 with which embodiments of the disclosed systems and methods, or portions thereof may be implemented, in accordance with various embodiments. For example, the illustrated computer system can be a local or remote computer system operatively connected to a control system for controlling or monitoring the systems and methods of the various embodiments herein. In various embodiments of the present teachings, computer system 1300 can include a bus 1302 or other communication mechanism for communicating information and a processor 1304 coupled with bus 1302 for processing information. In various embodiments, computer system 1300 can also include a memory, which can be a random-access memory (RAM) 1306 or other dynamic storage device, coupled to bus 1302 for determining instructions to be executed by processor 1304. Memory can also be used for storing temporary variables or other intermediate information during execution of instructions to be executed by processor 1304. In various embodiments, computer system 1300 can further include a read only memory (ROM) 1308 or other static storage device coupled to bus 1302 for storing static information and instructions for processor 1304. A storage device 1310, such as a magnetic disk or optical disk, can be provided and coupled to bus 1302 for storing information and instructions.

In various embodiments, computer system 1300 can be coupled via bus 1302 to a display 1312, such as a cathode ray tube (CRT) or liquid crystal display (LCD), for displaying information to a computer user. An input device 1314, including alphanumeric and other keys, can be coupled to bus 1302 for communication of information and command selections to processor 1304. Another type of user input device is a cursor control 1316, such as a mouse, a trackball or cursor direction keys for communicating direction information and command selections to processor 1304 and for controlling cursor movement on display 1312. This input device 1314 typically has two degrees of freedom in two axes, a first axis (i.e., x) and a second axis (i.e., y), that allows the device to specify positions in a plane. However, it should be understood that input devices 1314 allowing for 13-dimensional (x, y and z) cursor movement are also contemplated herein. In accordance with various embodiments, components 1312/1314/1316, together or individually, can make up a control system that connects the remaining components of the computer system to the systems herein and methods conducted on such systems, and controls execution of the methods and operation of the associated system.

Consistent with certain implementations of the present teachings, results can be provided by computer system 1300 in response to processor 1304 executing one or more sequences of one or more instructions contained in memory 1306. Such instructions can be read into memory 1306 from another computer-readable medium or computer-readable storage medium, such as storage device 1310. Execution of the sequences of instructions contained in memory 1306 can cause processor 1304 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 1304 for execution. Such a medium can take many forms, including but not limited to, non-volatile media, volatile media, and transmission media. Examples of non-volatile media can include, but are not limited to, dynamic memory, such as memory 1306. Examples of transmission media can include, but are not limited to, coaxial cables, copper wire, and fiber optics, including the wires that comprise bus 1302.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, another memory chip or cartridge, or any other tangible medium from which a computer can read.

In addition to computer-readable medium, instructions or data can be provided as signals on transmission media included in a communications apparatus or system to provide sequences of one or more instructions to processor 1304 of computer system 1300 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein, flow charts, diagrams and accompanying disclosure can be implemented using computer device/system 1300 as a standalone device or on a distributed network or shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1300, whereby processor 1304 would execute the analyses and determinations provided by these engines, subject to instructions provided by any one of, or a combination of, memory components 1306/1308/1310 and user input provided via input device 1314.

### Recitation of Embodiments

Embodiment 1. A method for performing multiplexed diagnostic testing, comprising: providing a sample comprising nucleic acid units to a plurality of channels of a multichannel device; introducing a unique set of one or more probes and one or more reporter moieties into each of the plurality of channels; detecting a first indicator readout from a first set of channels of the plurality of channels, wherein the first indicator readout is generated by at least one of the one or more reporter moieties as a result of interaction of a probe within the unique set with one of the nucleic acid units in the sample; generating a readout code for the sample based at least in part on the first indicator readout detected from the first set of channels of the plurality of channels; matching the generated readout code against a plurality of readout code entries of a lookup table, wherein the plurality of readout code entries of the lookup table represent a plurality of biological identities associated with the nucleic acid units; and generating a detection result of the multiplexed diagnostic testing for the sample based on the matched readout code.

Embodiment 2. The method of Embodiment 1, wherein the plurality of channels each comprise the same reporter moiety.

Embodiment 3. The method of Embodiment 1 or Embodiment 2, wherein the nucleic acid units comprise genomic information.

Embodiment 4. The method of any one of Embodiments 1-3, wherein the nucleic acid units comprise DNA, RNA, or a combination thereof.

Embodiment 5. The method of any one of Embodiments 1-4, wherein the plurality of biological identities are a plurality of pathogens and the nucleic acid units correspond to nucleic acids of the plurality of pathogens.

Embodiment 6. The method of Embodiment 4, wherein the nucleic acid units comprise microbial genomic DNA, viral genomic information, or a combination thereof.

Embodiment 7. The method of any one of Embodiments 1-3, wherein the nucleic acid units correspond to genomic nucleic acids of a subject.

Embodiment 8. The method of Embodiment 7, wherein the plurality of biological identities comprise genetic disease markers.

Embodiment 9. The method of Embodiment 7, wherein the plurality of biological identities comprise cancer-associated markers.

Embodiment 10. The method of any one of Embodiments 1-9, wherein the unique set of one or more probes are guide RNAs and each of the plurality of channels further includes a CRISPR type nuclease.

Embodiment 11. The method of Embodiment 10, wherein the CRISPR type nuclease is selected from a group consisting of Cas9, Cas12, Cas12a, MAD4, MAD7, Cas13,and Cas14.

Embodiment 12. The method of Embodiment 10, wherein one of the guide RNAs is engineered for interacting with one or more nucleic acid units associated with the plurality of biological identities.

Embodiment 13. The method of Embodiment 10, wherein one of the guide RNAs is engineered for interacting with two or more nucleic acid units associated with the plurality of biological identities.

Embodiment 14. The method of any one of Embodiments 1-13, wherein the unique set of one or more probes comprise PCR primers.

Embodiment 15. The method of any one of Embodiments 1-14, wherein the first indicator readout is selected from a group consisting of a visible signal, a fluorescent signal, a bioluminescent signal, a light-emitting signal, a radioactive emission, an electrical signal, and any combination thereof.

Embodiment 16. The method of any one of Embodiments 1-15, wherein the generating the readout code includes assigning one of two symbols of a bit for the first set of channels to generate a binary readout code.

Embodiment 17. The method of Embodiment 16, further comprising: failing to detect a second indicator readout from a second set of channels of the plurality of channels different from the first set of channels, wherein the generating the readout code comprises assigning the other symbol of the two symbols of the bit for the second set of channels to generate the binary readout code.

Embodiment 18. The method of Embodiment 16 or Embodiment 17, wherein: each readout code entry of the plurality of readout code entries corresponds to one of the 2ⁿ n-bit binary codes where n is a number of the plurality of channels; and the matching includes comparing the binary readout code to the one of the 2ⁿ n-bit binary codes.

Embodiment 19. The method of Embodiment 18, wherein the detection result indicates that the sample contains one of the plurality of biological identities represented by the one of the 2ⁿ n-bit binary codes when the comparison indicates that the binary readout code matches the one of the 2ⁿ n-bit binary codes.

Embodiment 20. The method of Embodiment 16 or Embodiment 17, wherein: each readout code entry of the plurality of readout code entries corresponds to one of binomial coefficient (n;k) number of binary codes where n is a number of the plurality of channels and k bits of each binary code share a same symbol different from symbol of any other bit of the binary code; and the matching includes comparing the binary readout code to the one of binomial coefficient (n;k) number of binary codes.

Embodiment 21. The method of Embodiment 20, wherein the detection result indicates that the sample contains one of the plurality of biological identities represented by the one of binomial coefficient (n;k) number of binary codes when the comparison indicates that the binary readout code matches the one of binomial coefficient (n;k) number of binary codes.

Embodiment 22. The method of Embodiment 20 or Embodiment 21, wherein k is equal to a natural number ranging from 1 to n/2.

Embodiment 23. The method of any one of Embodiment 20-22, further comprising: assigning high-prevalence biological identities to certain binary codes with higher distance away from other used codes, and/or assigning lower-prevalence biological identities to binary codes with lower distance away from other codes.

Embodiment 24. The method of Embodiment 16 or Embodiment 17, wherein: each readout code entry of the plurality of readout code entries belongs to a group of binary codes where trunk bits of each of the grouped binary codes located at same binary positions share same symbol; and the matching includes performing a first comparison of the trunk bits of the binary readout code located at the same binary positions to the trunk bits of any of the grouped binary codes.

Embodiment 25. The method of Embodiment 24, wherein: the first comparison indicates that the binary readout code belongs to the group of binary codes; the binary readout code entry corresponds to one of the grouped binary codes of the group of binary codes; and the matching further includes performing a second comparison of bits of the binary readout code other than the k bits of the binary readout code to bits of one of the grouped binary codes other than the k bits of the one of the grouped binary codes.

Embodiment 26. The method of Embodiment 25, wherein the diagnostic test result indicates that the sample contains one of the plurality of biological identities represented by the one of the grouped binary codes when the second comparison indicates that the bits of the binary readout code other than the k bits of the binary readout code match the bits of one of the grouped binary codes other than the k bits of the one of the grouped binary codes.

Embodiment 27. The method of any one of Embodiments 1-26, wherein the sample includes a bodily substance selected from a group consisting of blood, saliva, urine, feces, and mucus.

Embodiment 28. The method of any one of Embodiments 1-27, wherein the plurality of biological identities include pathogens from one or more of a bacterium, a fungus, a parasite and a virus.

Embodiment 29. The method of any one of Embodiments 1-28, further comprising: providing the sample to a first control channel and a second control channel of the multichannel device, wherein the first control channel is capable of identifying a first biological identity in the sample and wherein the second control channel is incapable of identifying the first biological identity in the sample.

Embodiment 30. The method of Embodiment 29, further comprising: validating the detection result when the first control channel identifies the first biological identity in the sample and/or the second control channel fails to identify the first biological identity in the sample.

Embodiment 31. The method of Embodiment 29, further comprising: invalidating the detection result when the first control channel fails to identify the first biological identity in the sample and/or the second control channel identifies the first biological identity in the sample.

Embodiment 32. The method of any one of Embodiments 1-31, wherein the sample comprises at least 100 different nucleic acid units and 500 different nucleic acid units.

Embodiment 33. The method of any one of Embodiments 1-32, wherein each of the plurality of channels comprises the unique set of one or more probes that are configured to detect at least 1 nucleic acid unit.

Embodiment 34. The method of any one of Embodiments 1-33, wherein the plurality of channels is configured to detect at least 2 biological identities.

Embodiment 35. The method of any one of Embodiments 1-34, wherein the detection result of the multiplexed diagnostic testing for the sample includes an identification of one biological identity from the plurality of biological identities listed in the plurality of readout code entries of the lookup table.

Embodiment 36. The method of any one of Embodiments 1-35, further comprising: applying a prevalence value of specific biological identities to the lookup table.

Embodiment 37. The method of Embodiment 36, wherein the specific biological identities comprise pathogens, the method further comprising: applying a rate of coinfections of specific biological identities to the lookup table.

Embodiment 38. A system for performing multiplexed diagnostic testing, comprising: a multichannel device having a plurality of channels configured to receive a sample comprising nucleic acid units, wherein each of the plurality of channels are configured to receive a unique set of one or more probes and one or more reporter moieties, and wherein the multichannel device is configured to detect a first indicator readout from a first set of channels of the plurality of channels, wherein the first indicator readout is generated by at least one of the one or more reporter moieties as a result of interaction of a probe within the unique set with one of the nucleic acid units in the sample; and a processor communicatively coupled to the multichannel device and configured to perform multiplexing operations comprising: generating a readout code for the sample based at least in part on the first indicator readout detected from the first set of channels of the plurality of channels; matching the generated readout code against a plurality of readout code entries of a lookup table, wherein the plurality of readout code entries of the lookup table represent a plurality of biological identities associated with the nucleic acid units; and generating a detection result of the multiplexed diagnostic testing for the sample based on the matched readout code.

Embodiment 39. The system of Embodiment 38, wherein the plurality of channels each comprise the same reporter moiety.

Embodiment 40. The system of Embodiment 38 or Embodiment 39, wherein the nucleic acid units comprise genomic information.

Embodiment 41. The system of any one of Embodiments 38-40, wherein the nucleic acid units comprise DNA, RNA, or a combination thereof.

Embodiment 42. The system of any one of Embodiments 38-41, wherein the plurality of biological identities are a plurality of pathogens and the nucleic acid units correspond to nucleic acids of the plurality of pathogens.

Embodiment 43. The system of Embodiment 42, wherein the nucleic acid units comprise microbial genomic DNA, viral genomic information, or a combination thereof.

Embodiment 44. The system of any one of Embodiments 38-40, wherein the nucleic acid units correspond to genomic nucleic acids of a subject.

Embodiment 45. The system of Embodiment 44, wherein the plurality of biological identities comprise genetic disease markers.

Embodiment 46. The system of Embodiment 44, wherein the plurality of biological identities comprise cancer-associated markers.

Embodiment 47. The system of any one of Embodiments 38-46, wherein the unique set of one or more probes are guide RNAs and each of the plurality of channels further includes a CRISPR type nuclease.

Embodiment 48. The system of Embodiment 47, wherein the CRISPR type nuclease is selected from a group consisting of Cas9, Cas12, Cas12a, MAD4, MAD7, Cas13, and Cas14.

Embodiment 49. The system of Embodiment 47, wherein one of the guide RNAs is engineered for interacting with one or more nucleic acid units associated with the plurality of biological identities.

Embodiment 50. The system of Embodiment 47, wherein one of the guide RNAs is engineered for interacting with two or more nucleic acid units associated with the plurality of biological identities.

Embodiment 51. The system of any one of Embodiments 38-50, wherein the unique set of one or more probes comprise PCR primers.

Embodiment 52. The system of any one of Embodiments 38-51, wherein the first indicator readout is selected from a group consisting of a visible signal, a fluorescent signal, a bioluminescent signal, a light-emitting signal, a radioactive emission, an electrical signal, and any combination thereof.

Embodiment 53. The system of any one of Embodiments 38-52, wherein the generating the readout code includes assigning one of two symbols of a bit for the first set of channels to generate a binary readout code.

Embodiment 54. The system of Embodiments 53, wherein the operations further comprising: failing to detect a second indicator readout from a second set of channels of the plurality of channels different from the first set of channels, wherein the generating the readout code comprises assigning the other symbol of the two symbols of the bit for the second set of channels to generate the binary readout code.

Embodiment 55. The system of Embodiment 53 or Embodiment 54, wherein: each readout code entry of the plurality of readout code entries corresponds to one of the 2ⁿ n-bit binary codes where n is a number of the plurality of channels; and the matching includes comparing the binary readout code to the one of the 2ⁿ n-bit binary codes.

Embodiment 56. The system of Embodiment 55, wherein the detection result indicates that the sample contains one of the plurality of biological identities represented by the one of the 2ⁿ n-bit binary codes when the comparison indicates that the binary readout code matches the one of the 2ⁿ n-bit binary codes.

Embodiment 57. The system of Embodiment 53 or Embodiment 54, wherein: each readout code entry of the plurality of readout code entries corresponds to one of binomial coefficient (n;k) number of binary codes where n is a number of the plurality of channels and k bits of each binary code share a same symbol different from symbol of any other bit of the binary code; and the matching includes comparing the binary readout code to the one of binomial coefficient (n;k) number of binary codes.

Embodiment 58. The system of Embodiment 57, wherein the detection result indicates that the sample contains one of the plurality of biological identities represented by the one of binomial coefficient (n;k) number of binary codes when the comparison indicates that the binary readout code matches the one of binomial coefficient (n;k) number of binary codes.

Embodiment 59. The system of Embodiment 57 or Embodiment 58, wherein k is equal to a natural number ranging from 1 to n/2.

Embodiment 60. The system of any one of Embodiments 57-59, wherein the operations further comprising: assigning high-prevalence biological identities to certain binary codes with higher distance away from other used codes, and/or assigning lower-prevalence biological identities to binary codes with lower distance away from other codes.

Embodiment 61. The system of Embodiment 53 or Embodiment 54, wherein: each readout code entry of the plurality of readout code entries belongs to a group of binary codes where trunk bits of each of the grouped binary codes located at same binary positions share same symbol; and the matching includes performing a first comparison of the trunk bits of the binary readout code located at the same binary positions to the trunk bits of any of the grouped binary codes.

Embodiment 62. The system of Embodiment 61, wherein: the first comparison indicates that the binary readout code belongs to the group of binary codes; the binary readout code entry corresponds to one of the grouped binary codes of the group of binary codes; and the matching further includes performing a second comparison of bits of the binary readout code other than the k bits of the binary readout code to bits of one of the grouped binary codes other than the k bits of the one of the grouped binary codes.

Embodiment 63. The system of Embodiment 62, wherein the diagnostic test result indicates that the sample contains one of the plurality of biological identities represented by the one of the grouped binary codes when the second comparison indicates that the bits of the binary readout code other than the k bits of the binary readout code match the bits of one of the grouped binary codes other than the k bits of the one of the grouped binary codes.

Embodiment 64. The system of any one of Embodiments 38-63, wherein the sample includes a bodily substance selected from a group consisting of blood, saliva, urine, feces, and mucus.

Embodiment 65. The system of any one of Embodiments 38-64, wherein the plurality of biological identities include pathogens from one or more of a bacterium, a fungus, a parasite and a virus.

Embodiment 66. The system of any one of Embodiments 38-65, wherein the operations further comprising: providing the sample to a first control channel and a second control channel of the multichannel device, wherein the first control channel is capable of identifying a first biological identity in the sample and wherein the second control channel is incapable of identifying the first biological identity in the sample.

Embodiment 67. The system of Embodiment 66, wherein the operations further comprising: validating the detection result when the first control channel identifies the first biological identity in the sample and/or the second control channel fails to identify the first biological identity in the sample.

Embodiment 68. The system of Embodiment 66, wherein the operations further comprising: invalidating the detection result when the first control channel fails to identify the first biological identity in the sample and/or the second control channel identifies the first biological identity in the sample.

Embodiment 69. The system of any one of Embodiments 38-68, wherein the sample comprises at least 100 different nucleic acid units and 500 different nucleic acid units.

Embodiment 70. The system of any one of Embodiments 38-69, wherein each of the plurality of channels comprises the unique set of one or more probes that are configured to detect at least 1 nucleic acid unit.

Embodiment 71. The system of any one of Embodiments 38-70, wherein the plurality of channels is configured to detect at least 2 biological identities.

Embodiment 72. The system of any one of Embodiments 38-71, wherein the detection result of the multiplexed diagnostic testing for the sample includes an identification of one biological identity from the plurality of biological identities listed in the plurality of readout code entries of the lookup table.

Embodiment 73. The system of any one of Embodiments 38-72, wherein the operations further comprising: applying a prevalence value of specific biological identities to the lookup table.

Embodiment 74. The system of Embodiment 73, wherein the specific biological identities comprise pathogens, the method further comprising: applying a rate of coinfections of specific biological identities to the lookup table.

Embodiment 75. A non-transitory machine-readable medium having stored thereon machine-readable instructions executable to cause a computing device to perform operations for generating a multiplexed diagnostic testing result, the operations comprising: receiving a first indicator readout from a first set of channels of a plurality of channels of a multichannel device, wherein the first indicator readout is generated by interaction of one of nucleic acid units in a sample with a probe within a unique set of one or more probes in the plurality of channels of the multichannel device; generating a readout code for the sample based at least in part on the first indicator readout detected from the first set of channels of the plurality of channels; matching the generated readout code against a plurality of readout code entries of a lookup table, wherein the plurality of readout code entries of the lookup table represent a plurality of biological identities associated with the nucleic acid units; and generating the multiplexed diagnostic testing result for the sample based on the matched readout code.

While the present teachings are described in conjunction with various embodiments, it is not intended that the present teachings be limited to such embodiments. On the contrary, the present teachings encompass various alternatives, modifications, and equivalents, as will be appreciated by those of skill in the art. The present description provides preferred exemplary embodiments, and is not intended to limit the scope, applicability, or configuration of the disclosure. Rather, the present description of the preferred exemplary embodiments will provide those skilled in the art with an enabling description for implementing various embodiments.

It is understood that various changes may be made in the function and arrangement of elements without departing from the spirit and scope as set forth in the appended claims. Thus, such modifications and variations are considered to be within the scope set forth in the appended claims. Further, the terms and expressions which have been employed are used as terms of description and not of limitation, and there is no intention in the use of such terms and expressions of excluding any equivalents of the features shown and described or portions thereof, but it is recognized that various modifications are possible within the scope of the claimed embodiments.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

Specific details are given in the present description to provide an understanding of the embodiments. However, it is understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

3. The method of claim 1, wherein the plurality of biological identities are a plurality of pathogens and the nucleic acid units correspond to nucleic acids of the plurality of pathogens.

4. The method of claim 1, wherein the plurality of biological identities comprises genetic disease markers.

5. The method of claim 1, wherein the plurality of biological identities comprises cancer-associated markers.

6. The method of claim 1, wherein the unique set of one or more probes are guide RNAs and each of the plurality of channels further includes a CRISPR type nuclease.

7. The method of claim 6, wherein the CRISPR type nuclease is selected from a group consisting of Cas9, Cas12, Cas12a, MAD4, MAD7, Cas 13, and Cas 14.

8. The method of claim 1, wherein the generating the readout code includes assigning one of two symbols of a bit for the first set of channels to generate a binary readout code.

9. The method of claim 8, further comprising:
failing to detect a second indicator readout from a second set of channels of the plurality of channels different from the first set of channels, wherein the generating the readout code comprises assigning the other symbol of the two symbols of the bit for the second set of channels to generate the binary readout code.

10. The method of claim 8, wherein:
a. each readout code entry of the plurality of readout code entries corresponds to one of the 2ⁿ n-bit binary codes where n is a number of the plurality of channels; and
b. the matching includes comparing the binary readout code to the one of the 2ⁿ n-bit binary codes.

11. The method of claim 10, wherein the detection result indicates that the sample contains one of the plurality of biological identities represented by the one of the 2ⁿ n-bit binary codes when the comparison indicates that the binary readout code matches the one of the 2ⁿ n-bit binary codes.

12. The method of claim 8, wherein:
each readout code entry of the plurality of readout code entries corresponds to one of binomial coefficient (n;k) number of binary codes where n is a number of the plurality of channels and k bits of each binary code share a same symbol different from symbol of any other bit of the binary code; and
the matching includes comparing the binary readout code to the one of binomial coefficient (n;k) number of binary codes.

13. The method of claim 12, wherein the detection result indicates that the sample contains one of the plurality of biological identities represented by the one of binomial coefficient (n;k) number of binary codes when the comparison indicates that the binary readout code matches the one of binomial coefficient (n;k) number of binary codes.

14. The method of claim 12, wherein k is equal to a natural number ranging from 1 to n/2.

15. The method of claim 12, further comprising:
assigning high-prevalence biological identities to certain binary codes with higher distance away from other used codes, and/or assigning lower-prevalence biological identities to binary codes with lower distance away from other codes.

16. The method of claim 8, wherein:
each readout code entry of the plurality of readout code entries belongs to a group of binary codes where trunk bits of each of the grouped binary codes located at same binary positions share same symbol; and
the matching includes performing a first comparison of the trunk bits of the binary readout code located at the same binary positions to the trunk bits of any of the grouped binary codes.

17. The method of claim 16, wherein:
the first comparison indicates that the binary readout code belongs to the group of binary codes;
the binary readout code entry corresponds to one of the grouped binary codes of the group of binary codes; and
the matching further includes performing a second comparison of bits of the binary readout code other than the k bits of the binary readout code to bits of one of the grouped binary codes other than the k bits of the one of the grouped binary codes.

18. The method of claim 17, wherein the diagnostic test result indicates that the sample contains one of the plurality of biological identities represented by the one of the grouped binary codes when the second comparison indicates that the bits of the binary readout code other than the k bits of the binary readout code match the bits of one of the grouped binary codes other than the k bits of the one of the grouped binary codes.

19. The method of claim 17, further comprising:
providing the sample to a first control channel and a second control channel of the multichannel device, wherein the first control channel is capable of identifying a first biological identity in the sample and
wherein the second control channel is incapable of identifying the first biological identity in the sample.

20. The method of claim 19, further comprising: validating the detection result when the first control channel identifies the first biological identity in the sample and/or the second control channel fails to identify the first biological identity in the sample.

21. The method of claim 19, further comprising: invalidating the detection result when the first control channel fails to identify the first biological identity in the sample and/or the second control channel identifies the first biological identity in the sample.

22. The method of claim 1, wherein the plurality of channels is configured to detect at least 2 biological identities.

23. The method of claim 1, wherein the detection result of the multiplexed diagnostic testing for the sample includes an identification of one biological identity from the plurality of biological identities listed in the plurality of readout code entries of the lookup table.

24. The method of claim 1, further comprising:
applying a prevalence value of specific biological identities to the lookup table.

25. The method of claim 1, wherein the specific biological identities comprise pathogens, the method further comprising:
applying a rate of coinfections of specific biological identities to the lookup table.

26. A system for performing multiplexed diagnostic testing, the system comprising:
a multichannel device having a plurality of channels configured to receive a sample comprising nucleic acid units, wherein each of the plurality of channels are configured to receive a unique set of one or more probes and one or more reporter moieties, and wherein the multichannel device is configured to detect a first indicator readout from a first set of channels of the plurality of channels, wherein the first indicator readout is generated by at least one of the one or more reporter moieties as a result of interaction of a probe within the unique set with one of the nucleic acid units in the sample; and
a processor communicatively coupled to the multichannel device and configured to perform multiplexing operations comprising:
generating a readout code for the sample based at least in part on the first indicator readout detected from the first set of channels of the plurality of channels;
matching the generated readout code against a plurality of readout code entries of a lookup table, wherein the plurality of readout code entries of the lookup table represent a plurality of biological identities associated with the nucleic acid units; and
generating a detection result of the multiplexed diagnostic testing for the sample based on the matched readout code.

27. The system of claim 26, wherein the plurality of biological identities are a plurality of pathogens and the nucleic acid units correspond to nucleic acids of the plurality of pathogens.

28. The system of claim 27, wherein the plurality of biological identities comprise genetic disease markers.

29. The system of claim 27, wherein the plurality of biological identities comprise cancer-associated markers.

30. The system of claim 26, wherein the unique set of one or more probes are guide RNAs and each of the plurality of channels further includes a CRISPR type nuclease.

31. The system of claim 30, wherein the CRISPR type nuclease is selected from a group consisting of Cas9, Cas12, Cas12a, MAD4, MAD7, Cas 13, and Cas14.

32. The system of claim 26, wherein the first indicator readout is selected from a group consisting of a visible signal, a fluorescent signal, a bioluminescent signal, a light-emitting signal, a radioactive emission, an electrical signal, and any combination thereof.

33. The system of claim 26, wherein the generating the readout code includes assigning one of two symbols of a bit for the first set of channels to generate a binary readout code.

34. The system of claim 33, wherein the operations further comprising failing to detect a second indicator readout from a second set of channels of the plurality of channels different from the first set of channels, wherein the generating the readout code comprises assigning the other symbol of the two symbols of the bit for the second set of channels to generate the binary readout code.

35. The system of claim 33, wherein:
each readout code entry of the plurality of readout code entries corresponds to one of the 2ⁿ n-bit binary codes where n is a number of the plurality of channels; and
the matching includes comparing the binary readout code to the one of the 2ⁿ n-bit binary codes.

36. The system of claim 35, wherein the detection result indicates that the sample contains one of the plurality of biological identities represented by the one of the 2ⁿ n-bit binary codes when the comparison indicates that the binary readout code matches the one of the 2ⁿ n-bit binary codes.

37. The system of claim 33, wherein:
each readout code entry of the plurality of readout code entries corresponds to one of binomial coefficient (n;k) number of binary codes where n is a number of the plurality of channels and k bits of each binary code share a same symbol different from symbol of any other bit of the binary code; and
the matching includes comparing the binary readout code to the one of binomial coefficient (n;k) number of binary codes.

38. The system of claim 37, wherein the detection result indicates that the sample contains one of the plurality of biological identities represented by the one of binomial coefficient (n;k) number of binary codes when the comparison indicates that the binary readout code matches the one of binomial coefficient (n;k) number of binary codes.

39. The system of claim 37, wherein k is equal to a natural number ranging from 1 to n/2.

40. The system of claim 37, wherein the operations further comprising:
assigning high-prevalence biological identities to certain binary codes with higher distance away from other used codes, and/or
assigning lower-prevalence biological identities to binary codes with lower distance away from other codes.

41. The system of claim 33, wherein:
each readout code entry of the plurality of readout code entries belongs to a group of binary codes where trunk bits of each of the grouped binary codes located at same binary positions share same symbol; and
the matching includes performing a first comparison of the trunk bits of the binary readout code located at the same binary positions to the trunk bits of any of the grouped binary codes.

42. The system of claim 41, wherein:
the first comparison indicates that the binary readout code belongs to the group of binary codes;
the binary readout code entry corresponds to one of the grouped binary codes of the group of binary codes; and
the matching further includes performing a second comparison of bits of the binary readout code other than the k bits of the binary readout code to bits of one of the grouped binary codes other than the k bits of the one of the grouped binary codes.

43. The system of claim 42, wherein the diagnostic test result indicates that the sample contains one of the plurality of biological identities represented by the one of the grouped binary codes when the second comparison indicates that the bits of the binary readout code other than the k bits of the binary readout code match the bits of one of the grouped binary codes other than the k bits of the one of the grouped binary codes.

44. The system of claim 26, wherein the operations further comprising: providing the sample to a first control channel and a second control channel of the multichannel device, wherein the first control channel is capable of identifying a first biological identity in the sample and wherein the second control channel is incapable of identifying the first biological identity in the sample.

45. The system of claim 44, wherein the operations further comprising:
validating the detection result when the first control channel identifies the first biological identity in the sample and/or the second control channel fails to identify the first biological identity in the sample.

46. The system of claim 44, wherein the operations further comprising:
invalidating the detection result when the first control channel fails to identify the first biological identity in the sample and/or the second control channel identifies the first biological identity in the sample.

47. The system of claim 26, wherein each of the plurality of channels comprises the unique set of one or more probes that are configured to detect at least 1 nucleic acid unit.

48. The system of claim 26, wherein the plurality of channels is configured to detect at least 2 biological identities.

49. The system of claim 26, wherein the detection result of the multiplexed diagnostic testing for the sample includes an identification of one biological identity from the plurality of biological identities listed in the plurality of readout code entries of the lookup table.

50. The system of claim 26, wherein the operations further comprising:
applying a prevalence value of specific biological identities to the lookup table.

51. The system of claim 50, wherein the specific biological identities comprise pathogens, the method further comprising:
applying a rate of confections of specific biological identities to the lookup table.

52. A non-transitory machine-readable medium having stored thereon machine-readable instructions executable to cause a computing device to perform operations for generating a multiplexed diagnostic testing result, the operations comprising:
a. receiving a first indicator readout from a first set of channels of a plurality of channels of a multichannel device, wherein the first indicator readout is generated by interaction of one of nucleic acid units in a sample with a probe within a unique set of one or more probes in the plurality of channels of the multichannel device;
b. generating a readout code for the sample based at least in part on the first indicator readout detected from the first set of channels of the plurality of channels;
c. matching the generated readout code against a plurality of readout code entries of a lookup table, wherein the plurality of readout code entries of the lookup table represent a plurality of biological identities associated with the nucleic acid units; and
d. generating the multiplexed diagnostic testing result for the sample based on the matched readout code.
